(19) United States
(12) Patent Application Publication
(10) Pub. No.: US 2022/0175330 A1
(43) Pub. Date: Jun. 9, 2022

(54) MEDICAL DIAGNOSTIC APPARATUS

(71) Applicant: CANON MEDICAL SYSTEMS CORPORATION, Otawara-shi (JP)

(72) Inventors: Yoshimasa KOBAYASHI, Nasushiobara (JP); Seiichirou NAGAI, Otawara (JP)

(73) Assignee: CANON MEDICAL SYSTEMS CORPORATION, Otawara-shi (JP)

(21) Appl. No.: 17/643,008

(22) Filed: Dec. 7, 2021

(30) Foreign Application Priority Data
Dec. 7, 2020 (JP) 2020-202850

(51) Int. Cl. A61B 6/00; A61L 2/24; A61L 2/10

(52) U.S. Cl. CPC A61B 6/4423; A61B 6/0435; A61L 2/10; A61L 2/24

(57) ABSTRACT

A medical diagnostic apparatus according to an embodiment includes a contact component, a deep ultraviolet source, and a processing circuitry. The contact component has a contact region that is contacted by a subject during a medical practice. The deep ultraviolet source is provided with the contact component to irradiate the contact region with deep ultraviolet rays. The processing circuitry is configured to control the deep ultraviolet source to irradiate the contact region with deep ultraviolet rays when the medical practice is not performed.

### CROSS-REFERENCE TO RELATED APPLICATIONS

This application is based upon and claims the benefit of priority from Japanese Patent Application No. 2020-202850, filed on Dec. 7, 2020; the entire contents of which are incorporated herein by reference.

### FIELD

Embodiments described herein relate generally to a medical diagnostic apparatus.

### BACKGROUND

Conventionally, various medical apparatuses such as diagnostic imaging apparatuses and treatment apparatuses have been used for medical practices such as examinations including image diagnosis and treatments. During imaging for image diagnosis or during treatment, the skin of the subject of these medical practices may come into direct contact with the medical apparatus. In this case, in order to prevent the spread of infection due to the transfer of bacteria and viruses via the medical apparatus among a plurality of subjects, a sterilization operation for the medical apparatus is performed each time the medical practice for one subject ends. As a sterilization operation, for example, there is a known technique for irradiating a medical apparatus with deep ultraviolet rays to inactivate a virus adhering to the medical apparatus.

However, when the medical apparatus has a complicated shape or it is difficult to move the medical apparatus, the sterilization operation is complicated as an apparatus that generates deep ultraviolet rays is located at an appropriate posture or the apparatus is moved to irradiate the medical apparatus with deep ultraviolet rays from various angles. This results in a decrease in the throughput of medical practices.

### BRIEF DESCRIPTION OF THE DRAWINGS

FIG. 1 is a block diagram illustrating an example of a configuration of an X-ray diagnostic apparatus according to an embodiment;

FIG. 2 is a diagram illustrating a portion contacted by a subject during imaging in the X-ray diagnostic apparatus of FIG. 1;

FIG. 3 is a cross-sectional view schematically illustrating an example of a configuration of an armrest of FIG. 2;

FIG. 4 is a front view schematically illustrating an example of a configuration of an imaging table of FIG. 2;

FIG. 5 is a top view schematically illustrating an example of the configuration of the imaging table of FIG. 2;

FIG. 6 is a flowchart illustrating an example of a sterilization control process according to the embodiment;

FIG. 7 is a flowchart illustrating an example of a sterilization management process according to the embodiment; and

FIG. 8 is a perspective view illustrating an example of a configuration of a sterilization system according to the embodiment.

### DETAILED DESCRIPTION

A medical diagnostic apparatus described in the embodiment below includes a contact component, a deep ultraviolet source, and a processing circuitry. The contact component has a contact region that is contacted by a subject during a medical practice. The deep ultraviolet source is provided with the contact component to irradiate the contact region with deep ultraviolet rays. The processing circuitry is configured to control the deep ultraviolet source to irradiate the contact region with deep ultraviolet rays when the medical practice is not performed.

Each embodiment will be described below in detail with reference to the drawings. In the description below, the parts denoted by the same reference numeral perform the same operation, and therefore duplicate descriptions will be omitted as appropriate.

### First Embodiment

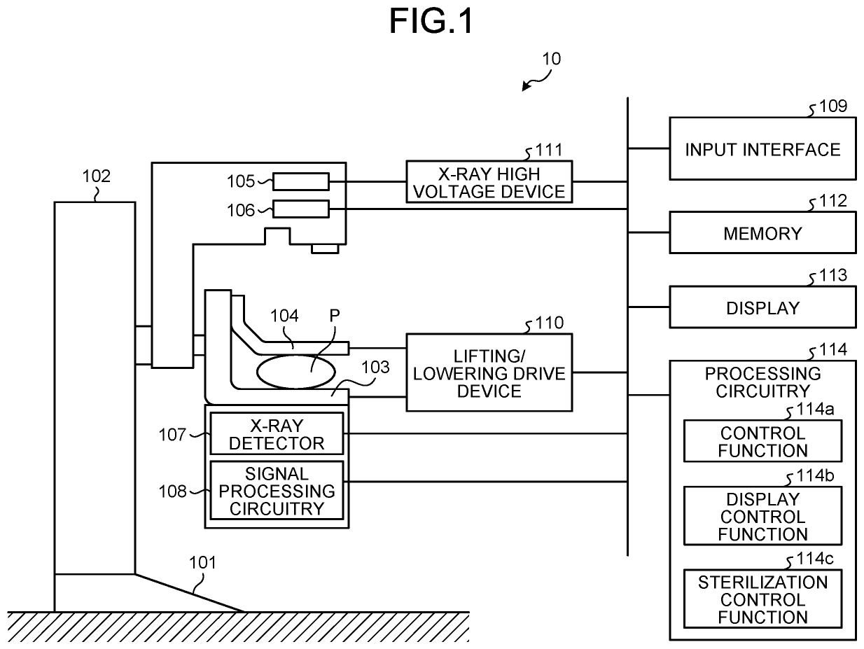

FIG. 1 is a diagram illustrating a configuration example of an X-ray diagnostic apparatus 10 according to an embodiment. Here, the X-ray diagnostic apparatus 10 is an example of a medical diagnostic apparatus. For the concrete descriptions, a case where the X-ray diagnostic apparatus 10 is a mammography apparatus will be described below as an example.

As illustrated in FIG. 1, the X-ray diagnostic apparatus 10 includes a base 101 and a stand 102. The stand 102 is provided to be upright on the base 101 and support an imaging table 103, a compression plate 104, an X-ray tube 105, an X-ray diaphragm device 106, an X-ray detector 107, and a signal processing circuitry 108. Here, the stand 102 supports the imaging table 103, the compression plate 104, the X-ray detector 107, and the signal processing circuitry 108 so as to move in a vertical direction.

The imaging table 103 is a table that supports a breast P of a subject (patient) and has a support surface where the breast P is placed. The compression plate 104 is provided above the imaging table 103 and is provided to face the imaging table 103 in parallel. Here, the compression plate 104 is provided to be movable in directions close to and away from the imaging table 103. For example, the compression plate 104 moves in a direction close to the imaging table 103 to press the breast P supported on the imaging table 103. The breast P is compressed by the compression plate 104 to be spread thin so that the mammary glands in the breast P are less overlapped.

The X-ray tube 105 is a vacuum tube including a cathode (filament) that generates thermoelectrons and an anode (target) that generates X-rays upon the collision of thermoelectrons. The X-ray tube 105 emits thermoelectrons from the cathode toward the anode by using a high voltage supplied from an X-ray high voltage device 111 to generate X-rays. Here, the X-ray tube 105 is configured to be movable to change the irradiation angle of the X-rays to the breast P.

The X-ray diaphragm device 106 is provided between the X-ray tube 105 and the compression plate 104 to control the X-rays generated by the X-ray tube 105. For example, the X-ray diaphragm device 106 includes a collimator that narrows the irradiation range of the X-rays and a filter that adjusts the X-rays.

The collimator of the X-ray diaphragm device 106 includes, for example, four slidable diaphragm blades and slides the diaphragm blades to narrow the X-rays generated by the X-ray tube 105 and irradiates the breast P with the X-rays. Here, the diaphragm blade is a plate-shaped member made of lead, or the like, and is provided near an X-ray irradiation port of the X-ray tube 105 to adjust the irradiation range of the X-rays.

For the purpose of reducing the exposure dose to the subject and improving the image quality of X-ray image data, the filter of the X-ray diaphragm device 106 changes the radiation quality of transmitted X-rays depending on the material or thickness thereof to reduce soft ray components that are easily absorbed by the subject and reduce high energy components that cause a decrease in image contrast. The filter changes the dose and irradiation range of X-rays depending on the material, thickness, position thereof, etc., and attenuates the X-rays such that the X-rays emitted to the breast P have a predetermined distribution.

For example, the X-ray diaphragm device 106 includes a drive mechanism such as a motor and an actuator and, under the control of a processing circuitry 114 described below, operates the drive mechanism to control the irradiation of the X-rays. For example, the X-ray diaphragm device 106 applies a drive voltage to the drive mechanism in response to a control signal received from the processing circuitry 114 to adjust the aperture of the diaphragm blades of the collimator and control the irradiation range of X-rays emitted to the breast P. For example, the X-ray diaphragm device 106 applies a drive voltage to the drive mechanism in response to a control signal received from the processing circuitry 114 to adjust the position of the filter and thus control the distribution of the dose of X-rays emitted to the breast P.

The X-ray detector 107 is, for example, an X-ray planar detector (flat panel detector: FPD) including detection elements arranged in a matrix. The X-ray detector 107 detects the X-rays emitted from the X-ray tube 105 and transmitted through the breast P and outputs the detection signal corresponding to the detected X-ray dose to the signal processing circuitry 108. The X-ray detector 107 may be an indirect-conversion type detector including a grid, a scintillator array, and an optical sensor array or a direct-conversion type detector including a semiconductor element that converts incident X-rays into electric signals.

For example, the X-ray detector 107 detects an X-ray pulse emitted from the X-ray tube 105 and generates the detection signal corresponding to the detected X-ray dose. Here, the X-ray detector 107 holds the generated detection signal. The X-ray detector 107 outputs the detection signal to the signal processing circuitry 108 after the emission of the X-ray pulse. Then, the signal processing circuitry 108 generates projection data based on the detection signal output from the X-ray detector 107 and stores the projection data in a memory 112.

As illustrated in FIG. 1, the X-ray diagnostic apparatus 10 includes an input interface 109, a lifting/lowering drive device 110, the X-ray high voltage device 111, the memory 112, a display 113, and the processing circuitry 114.

The input interface 109 receives various input operations from the operator, converts the received input operation into an electric signal, and outputs the electric signal to the processing circuitry 114. For example, the input interface 109 is implemented by a mouse, a keyboard, a trackball, a switch, a button, a joystick, a touch pad for performing an input operation with a touch on an operation surface, a touch screen having an integrated combination of a display screen and a touch pad, a non-contact input circuitry using an optical sensor, a voice input circuit, etc. The input interface 109 may be configured with a tablet terminal, or the like, capable of wireless communications with the processing circuitry 114. The input interface 109 is not limited to the one including a physical operating part such as a mouse and a keyboard. Examples of the input interface 109 also include an electric signal processing circuitry that receives the electric signal corresponding to an input operation from an external input device, which is provided separately from a main body of the X-ray diagnostic apparatus 10, and outputs the electric signal to the processing circuitry 114.

The lifting/lowering drive device 110 is coupled to the imaging table 103 and the compression plate 104. For example, the lifting/lowering drive device 110 lifts and lowers the imaging table 103 in the vertical direction. For example, the lifting/lowering drive device 110 lifts and lowers the compression plate 104 in the vertical direction (in a direction close to and away from the imaging table 103). For example, the lifting/lowering drive device 110 includes a drive mechanism such as a motor and an actuator and, under the control of the processing circuitry 114, operates the drive mechanism to control lifting/lowering of the imaging table 103 and the compression plate 104.

The X-ray high voltage device 111 supplies a high voltage to the X-ray tube 105 under the control of the processing circuitry 114. For example, the X-ray high voltage device 111 includes an electric circuitry such as a transformer and a rectifier, a high-voltage generation device that generates a high voltage to be applied to the X-ray tube 105, and an X-ray control device that controls an output voltage corresponding to the X-rays emitted by the X-ray tube 105. The high-voltage generation device may be of a transformer type or an inverter type.

The memory 112 is implemented by, for example, a semiconductor memory device such as a RAM or a flash memory, a hard disk, or an optical disk. For example, the memory 112 stores projection data generated by the signal processing circuitry 108 and mammography images such as mediolateral-oblique (MLO) images and craniocaudal (CC) images. For example, the memory 112 stores a program for a circuitry included in the X-ray diagnostic apparatus 10 to perform its function. The memory 112 may be implemented by a group of servers (cloud) connected to the X-ray diagnostic apparatus 10 via a network.

The display 113 presents various types of information. For example, the display 113 presents a GUI for receiving various instructions, various settings, and the like, from the operator via the input interface 109. The display 113 presents various types of image data collected for the breast P. For example, the display 113 is a liquid crystal display or a CRT display. The display 113 may be of a desktop type or may be configured with a tablet terminal, or the like, capable of wireless communications with the main body of the X-ray diagnostic apparatus 10.

The processing circuitry 114 performs a control function 114a, a display control function 114b, and a sterilization control function 114c to control the overall operation of the X-ray diagnostic apparatus 10.

For example, the processing circuitry 114 reads and executes the program corresponding to the control function 114a from the memory 112 to control various functions of the processing circuitry 114 based on the input operation received from the operator via the input interface 109.

The control function 114a controls collection of mammography images such as MLO images and CC images. Specifically, the control function 114a emits X-rays while fixing the positions of the imaging table 103 and the compression plate 104 in the MLO direction and the CC direction and keeping the constant X-ray irradiation angle with respect to the breast P to collect mammography images such as MLO images and CC images.

The control function 114a may also collect three-dimensional medical data. Specifically, first, the control function 114a executes tomosynthesis imaging on the breast P to collect a plurality of sets of projection data. Subsequently, the control function 114a performs correction processing such as logarithmic conversion processing, offset correction, sensitivity correction, and beam hardening correction on the collected projection data to generate corrected projection data. Then, the processing circuitry 114 reconstructs three-dimensional medical data based on the corrected projection data.

The processing circuitry 114 reads and executes the program corresponding to the display control function 114b from the memory 112 to present various types of image data on the display 113.

The processing circuitry 114 reads and executes the program corresponding to the sterilization control function 114c from the memory 112 to perform a sterilization control process. The sterilization control process will be described below.

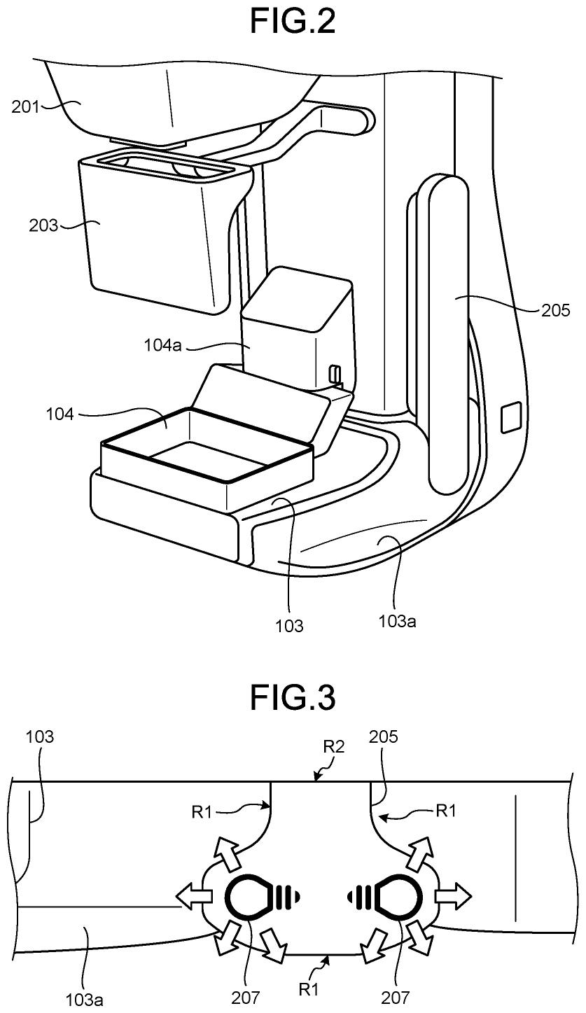

In the X-ray diagnostic apparatus 10 illustrated in FIG. 1, the memory 112 stores each processing function in the form of a program executable by a computer. The signal processing circuitry 108 and the processing circuitry 114 are processors that read and execute programs from the memory 112 to perform the functions corresponding to the respective programs. In other words, the signal processing circuitry 108 and the processing circuitry 114 having read the programs have the functions corresponding to the read programs. Although the single processing circuitry 114 executes the control function 114a, the display control function 114b, and the sterilization control function 114c in the description of FIG. 2, the processing circuitry 114 may be configured by a combination of a plurality of independent processors, and the function may be performed when each processor executes the program. Each processing function included in the processing circuitry 114 may be performed by being distributed or integrated into a single processing circuity or a plurality of processing circuitries.

The term “processor” used in the above description refers to, for example, a central processing unit (CPU), a graphics processing unit (GPU), or a circuit such as an application specific integrated circuit (ASIC) or a programmable logic device (e.g., simple programmable logic device (SPLD), complex programmable logic device (CPLD), and field programmable gate array (FPGA)). The processor reads and executes a program stored in the memory 112 to perform the function.

In the description of FIG. 1, the single memory 112 stores the program corresponding to each processing function. However, the embodiment is not limited thereto. For example, a configuration may be such that the memories 112 are dispersedly arranged and the processing circuitry 114 reads the corresponding program from the individual memory 112. Furthermore, a configuration may be such that, instead of storing the program in the memory 112, the program is directly installed in a circuitry of the processor. In this case, the processor reads and executes the program installed in the circuitry to perform the function.

The processing circuitry 114 may use a processor of an external device connected to the X-ray diagnostic apparatus 10 via a network to perform the function.

FIG. 2 is a diagram illustrating a portion contacted by the subject during imaging in the X-ray diagnostic apparatus 10 of FIG. 1. In the example described here, the breast P of the subject (patient) is captured by using the X-ray diagnostic apparatus 10 to collect a mammography image. Here, imaging with the X-ray diagnostic apparatus 10 is an example of a medical practice. The subject is an example of a subject of the medical practice.

The posture of the subject during imaging is defined by the arrangement of each unit of the X-ray diagnostic apparatus 10. As an example, while the subject holds armrests 205 provided on both side surfaces of an arm unit with both hands, the subject places the breast P on a support surface of the imaging table 103 and brings the face into contact with a face guard 203. The breast P of the subject is compressed by the imaging table 103 and the compression plate 104. The subject may also place the elbows on support tables 103a provided at both ends of the imaging table 103. As described above, during the imaging of the breast P by the X-ray diagnostic apparatus 10, the subject comes into contact with the support surface of the imaging table 103, the support table 103a, the compression plate 104, the face guard 203, and the armrest 205.

When imaging is conducted for a plurality of subjects by using the identical X-ray diagnostic apparatus 10, a sterilization operation is performed to sterilize the portion contacted by the subject each time the examination of each subject ends in order to prevent the spread of infection due to the transfer of bacteria and viruses through the portion contacted by the subject.

According to the present embodiment, sterilization refers to reducing the number of microorganisms, bacteria, and viruses or detoxifying microorganisms, bacteria, and viruses. Specifically, the sterilization according to the present embodiment includes removing and/or destroying at least a part of microorganisms and bacteria. Further, the sterilization according to the present embodiment includes removing and/or inactivating at least a part of viruses. As a sterilization operation, there is a known technique in which, for example, a portion contacted by the subject is irradiated with deep ultraviolet rays to inactivate viruses adhering to the portion contacted by the subject.

The compression plate 104 and the face guard 203, which are included in the portions contacted by the subject during imaging, are configured to be easily removed. Therefore, the compression plate 104 and the face guard 203 may be removed and thus easily sterilized even when a deep ultraviolet source is used, which generates deep ultraviolet rays outside the X-ray diagnostic apparatus 10.

The support table 103a is a portion contacted by the subject during imaging, but has a simple shape. Therefore, the support table 103a may be easily sterilized even when a deep ultraviolet source outside the X-ray diagnostic apparatus 10 is used.

An X-ray tube cover 201 covering and protecting the X-ray tube 105 and a compression plate support portion 104a supporting the compression plate 104 are portions that are not contacted by the subject during imaging and are portions that do not need to be sterilized for each subject.

However, when an external deep ultraviolet source is used for a portion having a complicated shape or a portion that is difficult to be removed, such as the imaging table 103 and the armrest 205, there is a need to emit deep ultraviolet rays from various angles, and the sterilization operation is complicated.

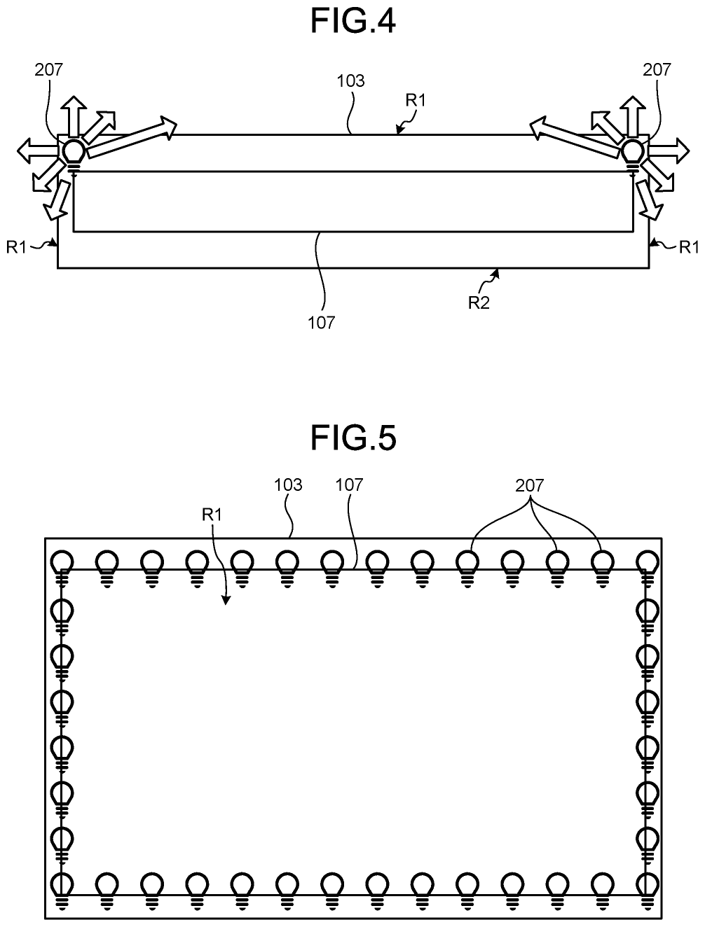

Therefore, the X-ray diagnostic apparatus 10 according to the present embodiment has a deep ultraviolet irradiation apparatus mounted therein. Specifically, in the X-ray diagnostic apparatus 10, the imaging table 103 and the armrest 205 each has a deep ultraviolet source 207, which generates deep ultraviolet rays, mounted inside. FIG. 3 is a cross-sectional view schematically illustrating an example of a configuration of the armrest 205 of FIG. 2. FIG. 4 is a front view schematically illustrating an example of a configuration of the imaging table 103 of FIG. 2. FIG. 5 is a top view schematically illustrating an example of the configuration of the imaging table 103 of FIG. 2.

As illustrated in FIGS. 3 to 5, the X-ray diagnostic apparatus 10 includes the plurality of deep ultraviolet sources 207. Each of the plurality of deep ultraviolet sources 207 generates deep ultraviolet rays having a wavelength in a predetermined ultraviolet range. For example, a light emitting diode (LED) may be used as each of the plurality of deep ultraviolet sources 207, but a mercury lamp or the like may be used as long as deep ultraviolet rays may be generated. The deep ultraviolet rays from each of the plurality of deep ultraviolet sources 207 are, for example, UV-C (wavelength: 200 nm to 280 nm).

The plurality of deep ultraviolet sources 207 is provided inside the armrest 205. Specifically, as illustrated in FIG. 3, each of the plurality of deep ultraviolet sources 207 is provided so as to irradiate a region R1, which is included in the armrest 205 and is likely to be contacted by the subject during imaging, with deep ultraviolet rays from inside the armrest 205.

In addition to the above-described X-ray detector 107, the plurality of deep ultraviolet sources 207 is further provided inside the imaging table 103. Specifically, as illustrated in FIGS. 4 and 5, each of the plurality of deep ultraviolet sources 207 is provided so as to irradiate the region R1, which is included in the imaging table 103 and is likely to be contacted by the subject during imaging, with deep ultraviolet rays from inside the imaging table 103. Each of the plurality of deep ultraviolet sources 207 is not provided on a path of X-rays incident on the detection surface of the X-ray detector 107 from the X-ray tube 105. Each of the plurality of deep ultraviolet sources 207 may be provided on a path of X-rays incident on a region other than the detection surface of the X-ray detector 107 from the X-ray tube 105.

A region R2, which is included in the armrest 205 and is not likely to be contacted by the subject during imaging, may be irradiated from inside the armrest 205 with deep ultraviolet rays from each of the plurality of deep ultraviolet sources 207. Similarly, the region R2, which is included in the imaging table 103 and is not likely to be contacted by the subject during imaging, may be irradiated from inside the imaging table 103 with deep ultraviolet rays from each of the plurality of deep ultraviolet sources 207.

The irradiation range of deep ultraviolet rays from each of the plurality of deep ultraviolet sources 207 may be, regardless of its location, adjusted as appropriate by a combination with an optical system that deflects deep ultraviolet rays.

The imaging table 103 and the armrest 205 are each formed of a member partially or entirely allowing transmission of deep ultraviolet rays. Specifically, in both the imaging table 103 and the armrest 205, at least the region R1, which is likely to be contacted by the subject during imaging, is formed of a member allowing transmission of deep ultraviolet rays. For example, glass such as quartz glass or deep ultraviolet transmitting glass may be used as a member allowing transmission of deep ultraviolet rays.

Here, an operation example of the X-ray diagnostic apparatus 10 will be described with reference to the drawing.

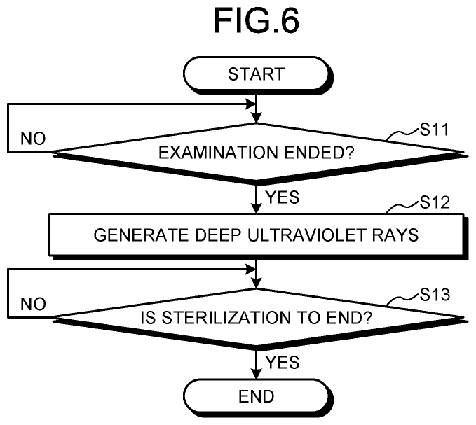

FIG. 6 is a flowchart illustrating an example of a sterilization control process according to the embodiment.

The sterilization control function 114c determines whether the examination has ended (Step S11). The sterilization control function 114c determines that the examination has ended when, for example, the input interface 109 has received an input operation for ending the examination from the operator. The flow of FIG. 6 stands by until it is determined that the examination has ended (Step S11: No), and when it is determined that the examination has ended (Step S11: Yes), proceeds to the process at Step S12.

The sterilization control function 114c causes the plurality of deep ultraviolet sources 207 to generate deep ultraviolet rays to start sterilization (Step S12). Subsequently, the sterilization control function 114c determines whether the sterilization is to end (Step S13). The sterilization control function 114c determines that the sterilization is to end when, for example, the elapsed time from the start of sterilization has reached a predetermined time. The process at Steps S12 and S13 is repeatedly performed until it is determined that the sterilization is to end (Step S13: No), and when it is determined that the sterilization is to end (Step S13: Yes), the flow of FIG. 6 ends.

Here, the predetermined time is preferably a time in which the virus adhering to the region R1 may be sufficiently inactivated. It is assumed that the predetermined time is previously set and stored in the memory 112, etc. The predetermined time may be set based on the intensity of deep ultraviolet rays generated by each of the plurality of deep ultraviolet sources 207, the distance from each of the plurality of deep ultraviolet sources 207 to the region R1, the transmittance of deep ultraviolet rays of the member forming the region R1, and the like.

The sterilization control function 114c may determine that the sterilization is to end when, for example, the input interface 109 has received an input operation for ending the sterilization from the operator. The sterilization control function 114c may determine that the sterilization is to end in accordance with not only the input operation for ending the sterilization but also the input operation for starting the examination for the subsequent subject, or the like. The sterilization control function 114c may determine that the sterilization is to end when the start time of the subsequent examination has reached.

The sterilization control function 114c may output the notification information for notifying the user that the sterilization is insufficient when it is determined that the sterilization is to end before the predetermined time has elapsed. In this case, the display control function 114b may cause the display 113 to present an image, characters, or the like, indicating that the sterilization is insufficient in accordance with the notification information.

As described above, in the X-ray diagnostic apparatus 10 according to the embodiment, the imaging table 103 and the armrest 205 each have the plurality of deep ultraviolet sources 207 mounted inside. The plurality of deep ultraviolet sources 207 is provided inside both the imaging table 103 and the armrest 205 so as to irradiate at least the region R1, which is likely to be contacted by the subject during imaging, with deep ultraviolet rays. In both the imaging table 103 and the armrest 205, at least the region R1, which is likely to be contacted by the subject during imaging, is formed of a member allowing transmission of deep ultraviolet rays.

This configuration allows the virus adhering to the region R1 on the outer surface, which is likely to be contacted by the subject during imaging, to be inactivated with the deep ultraviolet rays from the plurality of deep ultraviolet sources 207 provided inside. Therefore, it is possible to eliminate the need for a deep ultraviolet source outside the X-ray diagnostic apparatus 10 and to eliminate the effort of installing a deep ultraviolet source. Thus, with the technique according to the embodiment, it is possible to suppress a decrease in the throughput of an examination (medical practice) due to the sterilization operation with deep ultraviolet rays.

In the example described according to the first embodiment, the sterilization control process is performed while the compression plate 104 is removed, but the embodiment is not limited thereto. For example, the flow of FIG. 6 may be executed while the compression plate 104 is located close to the imaging table 103. In this case, the surface of the compression plate 104 on the side of the imaging table 103, i.e., the region that is likely to be contacted by the subject during imaging, may be sterilized with the deep ultraviolet rays from the plurality of deep ultraviolet sources 207 provided inside the imaging table 103.

In the example described according to the first embodiment, when the breast P is captured by the X-ray diagnostic apparatus 10, the subject comes into contact with the support surface of the imaging table 103, the support table 103a, the compression plate 104, the face guard 203, and the armrest 205, but the embodiment is not limited thereto. As the posture of the subject during imaging is defined by the arrangement of each unit of the X-ray diagnostic apparatus 10, the portion contacted by the subject during imaging may be different depending on the examination (medical practice) even with the identical apparatus. As an example, the subject comes into contact with the support table 103a during collection of MLO images, but does not come into contact with the support table 103a during collection of CC images.

Therefore, the sterilization control function 114c may selectively generate deep ultraviolet rays from the plurality of deep ultraviolet sources 207 during the sterilization control process. In this case, the sterilization control function 114c acquires the information indicating which medical practice was performed during the process at Step S11. The sterilization control function 114c refers to a table indicating the relationship between a medical practice and a sterilization target and stored in the memory 112 and selects the deep ultraviolet source 207, from which deep ultraviolet rays are to be generated, from the plurality of deep ultraviolet sources 207.

Each of the plurality of deep ultraviolet sources 207 may be configured to be movable inside the imaging table 103 and the armrest 205. In this case, the sterilization control function 114c further moves each of the plurality of deep ultraviolet sources 207 during the process at Step S12 of the sterilization control process. With this configuration, the number of deep ultraviolet sources 207 may be reduced. Further, during the sterilization control process, each of the plurality of deep ultraviolet sources 207 may be provided inside the imaging table 103 on a path of X-rays incident on the detection surface of the X-ray detector 107 from the X-ray tube 105. Accordingly, as compared with the arrangement illustrated in FIGS. 4 and 5, for example, deep ultraviolet rays may easily reach a central portion of the imaging table 103 so that the time required for the sterilization operation may be further shortened.

In the example described according to the first embodiment, the deep ultraviolet irradiation apparatus is mounted in the mammography apparatus, but the embodiment is not limited thereto. The deep ultraviolet irradiation apparatus according to the embodiment is applicable to various medical diagnostic imaging apparatuses, for example, X-ray diagnostic apparatus such as X-ray diagnostic apparatuses for circulatory organs, other than mammography apparatus, X-ray computed tomography (CT) apparatuses, and magnetic resonance imaging (MRI) apparatuses. The deep ultraviolet irradiation apparatus according to the embodiment is also applicable to not only medical diagnostic imaging apparatus but also treatment apparatuses.

As an example, in a medical apparatus, such as a medical diagnostic imaging apparatus or a treatment apparatus, having the deep ultraviolet irradiation apparatus according to the embodiment mounted therein, a top plate of a bed where the subject is placed during a medical practice (examination or treatment) has the plurality of deep ultraviolet sources 207 mounted inside. The plurality of deep ultraviolet sources 207 is provided inside the top plate so that at least the region that is likely to be contacted by the subject during a medical practice may be irradiated with the deep ultraviolet rays. In the top plate, at least the region that is likely to be contacted by the subject during a medical practice is formed of a member allowing transmission of deep ultraviolet rays. Even with this configuration, the same advantageous effect as that of the first embodiment may be obtained.

### Second Embodiment

Next, the X-ray diagnostic apparatus 10 according to a second embodiment will be described. The second embodiment is an example to prevent infection by managing sterilization information on each component. A difference from the first embodiment will be primarily described.

In the X-ray diagnostic apparatus 10 according to the present embodiment, the memory 112 stores sterilization information. Here, the sterilization information is information indicating whether each component, which is the target to be sterilized, of the X-ray diagnostic apparatus 10 is in a sterilized state or an unsterilized state after the last medical practice.

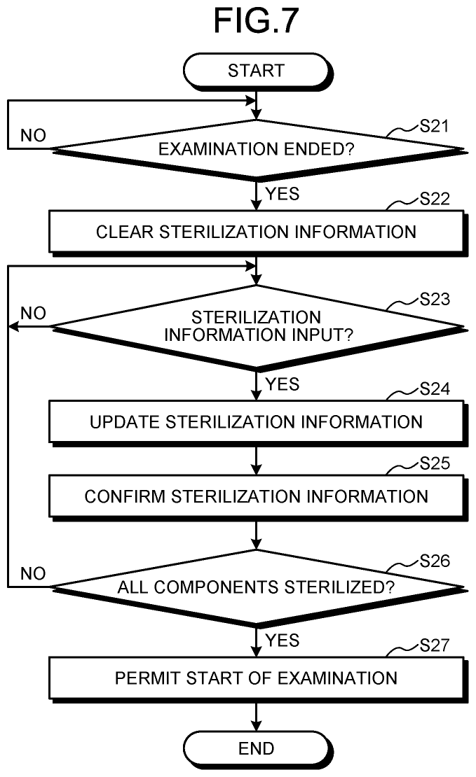

The processing circuitry 114 reads and executes the program corresponding to the sterilization control function 114c from the memory 112 to perform the sterilization control process. FIG. 7 is a flowchart illustrating an example of a sterilization management process according to the embodiment.

The sterilization control function 114c determines whether the examination has ended in the same manner as the process at Step S11 of the sterilization control process in FIG. 6 (Step S21). When it is not determined that the examination has ended (Step S21: No), the flow of FIG. 7 stands by.

Conversely, when it is determined that the examination has ended (Step S21: Yes), the sterilization control function 114c clears the sterilization information stored in the memory 112 and sets each component in the unsterilized state (Step S22).

It is assumed that the sterilization control process of FIG. 6 is started after the process at Step S22.

It is assumed that a component removed from the X-ray diagnostic apparatus 10 is sterilized during a flow different from the sterilization control process in FIG. 6. For example, components that may be separated from the X-ray diagnostic apparatus 10, such as the imaging table 103, the compression plate 104, and the face guard 203, may be housed in a sterilization dock to be sterilized.

The sterilization control function 114c determines whether the sterilization information has been input (Step S23). The sterilization control function 114c determines that the sterilization information has been input when, for example, the input interface 109 has received an input operation of the sterilization information from the operator. Alternatively, the sterilization control function 114c determines that the sterilization information has been input when sterilization completion information is received from the sterilization dock via a communication between the sterilization dock and the X-ray diagnostic apparatus 10. The operator may input the sterilization information via the input interface 109 with regard to the component that is housed in the sterilization dock to be sterilized.

When it is determined that the sterilization information has been input (Step S23: Yes), the sterilization control function 114c updates the sterilization information in the memory 112 based on the received input operation or the received sterilization completion information (Step S24). Subsequently, the sterilization control function 114c confirms the sterilization information in the memory 112 (Step S25) and determines whether all the components, which are the targets to be sterilized, of the X-ray diagnostic apparatus 10 have been sterilized after the last medical practice (Step S26).

Conversely, when it is not determined that the sterilization information has been input (Step S23: No) and when it is not determined that all the components have been sterilized (Step S26: No), the flow of FIG. 7 returns to the process at S23.

When it is determined that all the components have been sterilized (Step S26: Yes), the sterilization control function 114c permits the start of examination in response to the determination that all the components have been sterilized (Step S27). Subsequently, the flow of FIG. 7 ends. The state where the start of the examination has been permitted continues until the subsequent examination is started.

As described above, in the X-ray diagnostic apparatus 10 according to the present embodiment, by using the sterilization information indicating the sterilized state of each unit, the examination (medical practice) for the subsequent subject is permitted after all the components have been sterilized. With this configuration, when the sterilization of each unit has not completed, the examination of the subsequent subject is not started, and therefore the infection via the X-ray diagnostic apparatus 10 may be prevented.

### Third Embodiment

Next, the X-ray diagnostic apparatus 10 according to a third embodiment will be described. According to each of the above-described embodiments, the X-ray diagnostic apparatus 10 having the deep ultraviolet irradiation apparatus mounted therein is described as an example, but the embodiment is not limited thereto. The deep ultraviolet irradiation apparatus may also be configured as an apparatus independent of the X-ray diagnostic apparatus 10. A difference from the first embodiment will be primarily described.

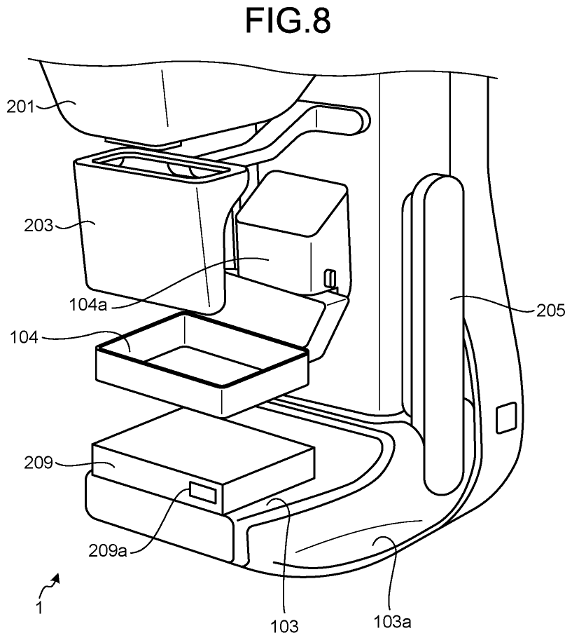

FIG. 8 is a perspective view illustrating an example of a configuration of a sterilization system 1 according to the embodiment. As illustrated in FIG. 8, the sterilization system 1 includes the X-ray diagnostic apparatus 10 and a deep ultraviolet irradiation apparatus 209.

As illustrated in FIG. 8, a chassis of the deep ultraviolet irradiation apparatus 209 has a shape that is sandwiched between the imaging table 103 and the compression plate 104. The chassis has, for example, a plate shape.

The plurality of deep ultraviolet sources 207 is mounted inside the chassis of the deep ultraviolet irradiation apparatus 209. The plurality of deep ultraviolet sources 207 is provided inside the chassis so as to irradiate at least the region R1, which is likely to be contacted by the subject during imaging, with deep ultraviolet rays. Specifically, the plurality of deep ultraviolet sources 207 is provided inside the chassis so as to generate the deep ultraviolet rays toward the surface opposed to the support surface of the imaging table 103 and the surface opposed to the lower surface of the compression plate 104 when the deep ultraviolet irradiation apparatus 209 is sandwiched between the imaging table 103 and the compression plate 104.

Further, the surface opposed to the support surface of the imaging table 103 and the surface opposed to the lower surface of the compression plate 104, of the chassis of the deep ultraviolet irradiation apparatus 209 when sandwiched between the imaging table 103 and the compression plate 104, are formed of a member allowing transmission of deep ultraviolet rays.

The deep ultraviolet irradiation apparatus 209 includes the processing circuitry 114, which performs the sterilization control function 114c, and an input/output unit 209a including, for example, a switch and an LED.

The sterilization control function 114c starts irradiation of deep ultraviolet rays from the plurality of deep ultraviolet sources 207 when the input/output unit 209a has received an input operation for starting sterilization from the operator. The deep ultraviolet irradiation apparatus 209 may be configured to detect compression by the imaging table 103 and the compression plate 104. In this case, the irradiation of deep ultraviolet rays may be started when the compression is detected.

The sterilization control function 114c notifies the operator that the deep ultraviolet rays are being generated by display using the LED of the input/output unit 209a so that it is possible to visually recognize that the deep ultraviolet rays are being generated from outside the apparatus. The notification may be made by sound.

The sterilization control function 114c stops the irradiation of the deep ultraviolet rays when a predetermined time has elapsed from the start of the irradiation of the deep ultraviolet rays, and the input/output unit 209a gives a notification that the sterilization has completed. The notification may be displayed by the LED of the input/output unit 209a or may be given by sound.

The operation of the deep ultraviolet irradiation apparatus 209 may be controlled by a control signal transmitted and received via a communication with the X-ray diagnostic apparatus 10. The above-described notification by the input/output unit 209a may be executed by the X-ray diagnostic apparatus 10.

The chassis of the deep ultraviolet irradiation apparatus 209 may have a shape other than a plate shape as long as the chassis has a shape sandwiched between the imaging table 103 and the compression plate 104. For example, the chassis of the deep ultraviolet irradiation apparatus 209 may include a plate portion having a shape sandwiched between the imaging table 103 and the compression plate 104 and a wall portion having a shape covering a side surface of the compression plate 104. In this case, the deep ultraviolet irradiation apparatus 209 may also irradiate a side surface portion of the compression plate 104 with the deep ultraviolet rays.

In the example described according to the present embodiment, in order to sterilize the imaging table 103 and the compression plate 104, the deep ultraviolet irradiation apparatus 209 has a shape (substantially the shape of a cuboid) sandwiched between the imaging table 103 and the compression plate 104, but the embodiment is not limited thereto. The shape of the deep ultraviolet irradiation apparatus 209 may be determined as appropriate in accordance with the shape of a component to be sterilized. For example, the deep ultraviolet irradiation apparatus 209, which sterilizes the armrest 205, has a shape that conforms to the shape of the outer surface of the region R1, which is likely to be contacted by the subject during imaging, of the armrest 205.

As described above, the sterilization system according to the present embodiment uses, for example, the deep ultraviolet irradiation apparatus 209 (deep ultraviolet generation apparatus) having a shape sandwiched between the plate-like imaging table 103 and the compression plate 104. Accordingly, a primary portion of the X-ray diagnostic apparatus 10, which is contacted by the subject when the breast P is captured, may be sterilized without removing the primary portion. Although the deep ultraviolet rays are largely attenuated in the air, the shape sandwiched between the imaging table 103 and the compression plate 104 may reduce uneven sterilization. The technique according to the present embodiment may be combined with the technique according to not only the first embodiment but also the second embodiment.

According to at least one of the embodiments described above, it is possible to suppress a decrease in the throughput of a medical practice due to a sterilization operation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical diagnostic apparatus comprising:
a contact component having a contact region that is contacted by a subject during a medical practice;
a deep ultraviolet source that is provided with the contact component to irradiate the contact region with deep ultraviolet rays; and
a processing circuitry configured to control the deep ultraviolet source to irradiate the contact region with deep ultraviolet rays when the medical practice is not performed.

2. The medical diagnostic apparatus according to claim 1, wherein
the contact region of the contact component is formed of a member allowing transmission of deep ultraviolet rays, and
the deep ultraviolet source is provided inside the contact component to irradiate the contact region with deep ultraviolet rays via the member from inside the component.

3. The medical diagnostic apparatus according to claim 1, wherein the processing circuitry is further configured to start to generate deep ultraviolet rays from the deep ultraviolet source when the medical practice ends.

4. The medical diagnostic apparatus according to claim 2, wherein
the deep ultraviolet source is configured to be movable inside the contact component, and
the processing circuitry is further configured to cause the deep ultraviolet source to irradiate the contact region with the deep ultraviolet rays while the deep ultraviolet source is moved inside the contact component.

5. The medical diagnostic apparatus according to claim 1, wherein the processing circuitry is further configured to determine whether to irradiate the contact component with the deep ultraviolet rays in accordance with the medical practice.

6. The medical diagnostic apparatus according to claim 1, further comprising a memory that stores sterilization information indicating whether the contact component is in either a sterilized state or an unsterilized state, wherein
the processing circuitry is further configured to update the sterilization information on the contact component to the unsterilized state when the medical practice ends and refrains from allowing start of a subsequent medical practice until the sterilization information on the contact component is updated to the sterilized state.

7. The medical diagnostic apparatus according to claim 1, further comprising an external deep ultraviolet irradiation apparatus including:
a chassis that has a shape that conforms to a shape of an outer surface of the contact region of the contact component and is provided to come into contact with the outer surface of the contact component at the contact region; and
an external deep ultraviolet source that is provided inside the chassis to irradiate the contact region of the contact component with deep ultraviolet rays via the chassis from inside the chassis.

8. The medical diagnostic apparatus according to claim 1, wherein
the contact component is a component that is included in components of the medical diagnostic apparatus and that defines a position or posture of the subject during the medical practice.
